(19) United States
(12) Patent Application Publication
(10) Pub. No.: US 2025/0041488 A1
(43) Pub. Date: Feb. 6, 2025

(54) HYDROGEL MICROPARTICLE-BASED SOFT TISSUE FILLERS

(71) Applicant: Likarda, LLC, Kansas City, MO (US)

(72) Inventors: Megan E. HAMILTON, Kansas City, MO (US); Stephen HARRINGTON, Kansas City, MO (US); Karthik RAMACHANDRAN, Kansas City, MO (US); Lisa STEHNO-BITTEL, Kansas City, MO (US)

(21) Appl. No.: 18/717,569

(22) PCT Filed: Dec. 9, 2022
(86) PCT No.: PCT/US2022/081274

§ 371 Date: Jun. 7, 2024

Related U.S. Application Data
(60) Provisional application No. 63/287,769, filed on Dec. 9, 2021.

(51) Int. Cl. A61L 27/52; A61L 27/16; A61L 27/18; A61L 27/20; A61L 27/22; A61L 27/24; A61L 27/54

(52) U.S. Cl. CPC A61L 27/52; A61L 27/16; A61L 27/18; A61L 27/20; A61L 27/222; A61L 27/24; A61L 27/54; A61L 2400/06; A61L 2430/34

(57) ABSTRACT

Methods, compositions, and kits for aesthetic or cosmetic soft tissue filling in a human subject. The compositions and methods are characterized as a suspension comprising a plurality of porous viscoelastic solid hydrogel microparticles dispersed in a carrier vehicle, wherein each microparticle is a self-sustaining body having an outer surface and a defined/discrete 3-dimensional shape and geometry, wherein the microparticles are substantially free of any cells, tissue, or therapeutic compounds.

### CROSS-REFERENCE TO RELATED APPLICATIONS

The present application claims the priority benefit of U.S. Provisional Patent Application Ser. No. 63/287,769, filed Dec. 9, 2021, entitled HYDROGEL MICROPARTICLE-BASED DERMAL FILLERS, incorporated by reference in its entirety herein.

### BACKGROUND

### Field of Invention

The present disclosure relates to fully crosslinked microparticles made of a variety of hydrogels for use as dermal or other soft tissue fillers.

### DESCRIPTION OF THE FIELD

Over the past 100 years, humans have tried to improve their appearance including the injection of various materials into the face and other body parts to increase volume and reduce wrinkles. In fact, there are reports of the injection of autologous fat into the face dating back to the 1890s. Later paraffin was injected into the faces of women in Europe. Reports of long-term complications from paraffin injections began in the early 1900s, but it was still used as a volume enhancer in some parts of Asia well into the 1950s. Other materials once used in the pursuit of beauty included injections of Polytetrafluoroethylene, silicone oil, and bovine collagen.

The field took a major leap when hyaluronic acid (HA) first approved by the FDA for injections in 2003. Hyaluronic acid is a polysaccharide that consists of repeating monomers (glucuronic acid and N-acetylglucosamine disaccharide units) linked together in a linear fashion through β-1,4 glycosidic bonds. The importance of HA as a cosmetic filler mitigated two problems with the previous fillers. First, it was easy to inject. Second, strong inflammatory reactions were less frequent. Additional beneficial characteristics were the ability to be stored at room temperature with a long shelf life, noncarcinogenic, and the effects lasted for months. HA fillers are typically partially crosslinked using 1,4-butanediol diglycidyl ether (BDDE) as the crosslinking agent to increase their durability.

There remains a need in the art for alternative and improved dermal filler materials.

### SUMMARY

The present disclosure describes the first approach of using fully crosslinked viscoelastic hydrogel microparticles which can be delivered as soft tissue fillers to improve aesthetic or cosmetic features, including filling wrinkles, creating volume, correcting mild deformities secondary to aging, acne, injury or surgery, treating lack of skin elasticity, treating lack of skin tautness, and correction of soft tissue defects or voids. As such, soft tissue filler placement can be done in the superficial dermis, mid-dermis, sub-dermal/subcutaneous layers, as well as into and just above the periosteum/periosteal layers. In contrast to the current uncrosslinked or partially crosslinked solutions or pastes, the crosslinked microparticles remain in the location of implantation, where placed, for a longer period of time, thus providing longer duration of cosmetic or aesthetic corrections.

Current hyaluronic acid fillers are comprised of a viscous liquid solution that is partially crosslinked and provides short duration effects. Table 3 summarizes the current commercially available HA-based fillers compared to the present fully crosslinked viscoelastic hydrogel microparticle. In comparing the crosslinking percentages for marketed products, RESTYLANE® has less crosslinking and a lower particle size, resulting in a less durable product, compared to JUVEDERM® Ultra with higher crosslinking and a slower rate of degradation. The Table illustrates the higher molecular weight of the hydrogel microparticles, given that an individual crosslinked microparticle of the invention can be considered a single molecule. In addition, the crosslinked microparticles have significantly higher crosslinking percentages. Table 4 summarizes some of the same information but compares the characteristics of the microparticles comprised of polyethylene glycol (PEG) to other non-HA hydrogels sold in the human cosmetic filler market. Calcium hydroxylapatite (CaHA) is another injectable dermal filler material (RADIESSE®) that contains uniform CaHA microspheres suspended in an aqueous carboxymethylcellulose gel carrier, and works by stimulating collagen production at the site of injection. However, this ceramic/mineral based filler cannot be dissolved if the patient does not like the results or if there is an unwanted interaction or side effect.

Described herein are methods, compositions, and kits for aesthetic or cosmetic soft tissue filling in a human subject. In one or more embodiment, methods are disclosed which comprise placing or introducing into a site of implantation in a subject in need thereof, a suspension comprising a plurality of porous viscoelastic solid hydrogel microparticles dispersed in a carrier vehicle. Each microparticle is a self-sustaining body having an outer surface and a defined/discrete 3-dimensional shape and geometry, wherein the microparticles are substantially free of any cells, tissue, or therapeutic compounds. In one or more embodiments, the suspension is placed into the superficial dermal layers, mid-dermal layers, sub-dermal/subcutaneous layers, facia, muscle, or periosteal layers of the subject.

Also described herein are kits for cosmetic or aesthetic soft tissue filling. The kits comprise a plurality of porous elastic solid hydrogel microparticles, wherein each microparticle is a self-sustaining body having an outer surface and a defined/discrete 3-dimensional shape and geometry, and substantially free of any cells, tissue, or therapeutic compounds, and instructions for administering the same.

Various compositions are also described herein for use in aesthetic or cosmetic soft tissue filling in a human subject. The compositions are generally characterized as suspensions comprising a plurality of porous viscoelastic solid hydrogel microparticles dispersed in a carrier vehicle, wherein each microparticle is a self-sustaining body having an outer surface and a defined/discrete 3-dimensional shape and geometry, wherein the microparticles are substantially free of any cells, tissue, or therapeutic compounds.

Uses of such compositions are also contemplated herein for aesthetic or cosmetic soft tissue filling in a human subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 contains images of microparticles of different defined geometric shapes that can be injected as a dermal filler: A) spherical shapes, B) teardrops, C) ovals, D) tubule-like shapes; E) mixture of shapes.

FIG. 2 contains images illustrating the viscoelastic solid fragments of hydrogel that result from forcing the microparticles through an opening smaller than the diameter of the microparticles.

FIG. 3 images illustrating the variety of sizes that can be made as crosslinked particles. A) Average diameters of 2040+21 μm, B) Average diameters of 1214+46 μm, C) Average diameters of 361+19 μm, D) Mixture of sizes.

FIG. 4 provides examples of different materials that can be used to make the microparticles as indicated in the figure.

FIG. 5 is a schematic providing one example of how the microparticles can be modified after crosslinking with complimentary molecules.

FIG. 6 contains images showing that the hydrogels can be fluorescently labeled to track their location and duration in the body.

FIG. 7 contains images of hydrogel microparticles exposed to solute-controlled size reduction.

FIG. 8 summarizes the changes in diameter of three different hydrogel chemistries when exposed to solute-controlled size reduction.

FIG. 9 summarizes the changes in microparticle volume of three different hydrogel chemistries when exposed to solute-controlled size reduction.

FIG. 10 illustrates the normalized reduction in sphere volume to different concentrations of the solute.

FIG. 11 illustrates the reduction in microparticle diameters (light gray) and the return to the original size (dark gray) for microparticles placed in different concentrations of solute.

FIG. 12 illustrates two methods of microparticle placement under the skin as a bolus injection or dispersed.

FIG. 13 contains images illustrating the biocompatibility of three different formulations of microparticles injected near or in the omentum of healthy rats.

FIG. 14 illustrates the lack of cell inflammatory or fibrotic cell adhesion to unattached microparticles after 2 weeks in the rat peritoneum.

FIG. 15 compares the in vivo degradation of one of the microparticle formulations compared to a non-biocompatible material.

FIG. 16 contains images of microparticles using a specific hydrogel formulation as they are degraded over time by enzymes.

FIG. 17 contains images of microparticles chemically degraded on demand.

FIG. 18A describes potential degradation pathways to dissolve fully crosslinked microparticles along with the potential biproducts.

FIG. 18B describes potential degradation pathways to dissolve fully crosslinked microparticles along with the potential biproducts.

FIG. 19 contains images of thiolated HA microparticles that were stored at room temperature in water for over 4 years and then imaged, showing that there was no visible loss of structure in the microparticles.

FIG. 20 are images of microparticles (PEG-MAL) prior to freezing in liquid nitrogen and thawing showing intact microparticles following the cryopreservation.

### DETAILED DESCRIPTION

The microparticles comprise, consist essentially, or even consist of a 3-dimensional matrix of fully crosslinked (via covalent or strongly ionic crosslinks) hydrogel polymer compounds, such that the resulting crosslinked microparticle body is characterized as a porous elastic solid (hydrogel) wherein elastic deformation is reversible. In other words, the microparticle is semi-rigid, but resilient such that the microparticle can flex under load and returns to its original shape after the load is removed. Suitable hydrogel precursor compounds for use in forming the microparticles include hydrogel-forming polymers, oligomers, and/or monomers, and as such are capable of forming a crosslinked or network structure or matrix through covalent or strongly ionic crosslinking, wherein liquid may be retained, suspended, entrapped, and/or encapsulated within the interstitial spaces or pores of the resulting elastic gelled structure or matrix body (hydrogel). Preferably, the precursor compounds are functionalized hydrogel-forming polymers, oligomers, and/or monomers, which means the native polymer, oligomer, or monomer backbone has been modified chemically to include a plurality of chemically reactive groups (i.e., functional groups) through which the covalent or ionic bonds are formed (i.e., crosslinked) during formation of the hydrogel matrix. In this manner, the fully crosslinked hydrogel particles obtained after the sol-gel transition do not rely on molecular entanglements, van der Waals forces, or other weak molecular interactions, but instead are characterized by covalent or strong ionic bonds forming an irreversible, insoluble hydrogel having a crosslinked network or matrix throughout the whole substance of the particle.

The microparticles are 3-dimensional self-sustaining bodies meaning that they retain their particular viscoelastic shape without an external support structure once that shape is formed and are not susceptible to deformation or creep merely due to its own weight or gravity. In other words, the self-sustaining body is not permanently deformable, or flowable, like a jelly, putty, or paste, but is resilient, such that the matrix body may temporarily yield or deform under force, and unless fractured, will return to the original shape upon removal of the force. The microparticles will typically have a particle size of less than about 2,000 μm, preferably less than about 1,500 μm, but greater than about 30 μm, preferably greater than about 100 m. As used herein, the “particle size” refers to the maximum surface-to-surface dimension (e.g., diameter in the case of somewhat spherical bodies). As discussed in more detail below, the microparticles are preferably rounded 3-dimensional bodies, with an aspect ratio of at least 0.5, preferably at least 0.7, and preferably about 1. As used herein the “aspect ratio” refers to the ratio of the maximum surface-to-surface dimension (i.e., particle size) to the minimum surface-to-surface dimension of each particle.

The hydrogel microparticles are not merely “particulates” or random aggregates, but can also be characterized as microspheres, microbeads, or hydrogel microparticles. Depending upon the polymer system used, the microparticles can generally be characterized by a Young's elastic modulus ranging from about 1,000 Pa to about 2.5 MPa (megapascal), more preferably about 10,000 Pa to about 2 MPa, more preferably from about 25,000 Pa to about 1 MPa, more preferably from about 50,000 Pa to about 0.5 MPa. Although resilient and having an elastic component, the microparticles, under sufficient application of force, may fracture or break into smaller pieces; however, such subsequent pieces would still comprise the 3-dimensional polymer matrix, albeit as smaller-sized bodies. In other words, the hydrogel microparticles are not shear thinning, nor is the matrix body itself susceptible to dissolution or dilution in a solvent system; although swelling of individual matrix bodies may occur depending upon the carrier or vehicle system (i.e., as liquid moves into the interstitial spaces or pores of the matrix). In one or more embodiments, the hydrogel microparticles can be characterized as irreversible hydrogels, meaning that once fractured, the matrix crosslinks will not reform or otherwise recover or self-heal.

Exemplary polymer precursor compounds used for forming the microparticles include fast-gelling natural polymers that form strong matrices, such as alginate, as well as slow-gelling polymer precursors selected from the group consisting of branched or unbranched hyaluronic acid, branched or unbranched functionalized hyaluronic acid, branched or unbranched functionalized polyethylene glycol, hyaluronan, fibrin, chitosan, collagen, polylactic acid, poly(L-lactic acid), polylactic-co-glycolic acid, polycaprolactone, polyvinyl alcohol, and combinations thereof.

The matrix can further comprise crosslinking agents (crosslinked with the polymer compounds) selected from the group consisting of dithiothreitol, branched or unbranched functionalized polyethylene glycol, dithiols, ethylene glycol bis-mercaptoacetate, and combinations thereof. Exemplary functionalized polyethylene glycols include, without limitation, polyethylene glycol dithiol, polyethylene glycol diacrylate, polyethylene glycol divinyl sulfone, polyethylene glycol dimaleimide, and combinations thereof. In one or more embodiments, the polymer precursor solution is substantially free of 1,4-butanediol diglycidyl ether (BDDE).

The matrix could be homogenous comprising one type of covalently crosslinked polymer backbone (with or without an additional crosslinker of a different polymer type). The matrix could also be heterogenous comprising a mixture of two or more polymer precursors, such as a combination of high (≥500 kDA, preferably ≥100 kDA) and low (≤100 kDA, preferably ≤50 kDa) molecular weight polymer precursors, and/or a mixture of two or more crosslinking agents.

In general, the amount of polymer precursor included in the precursor solution will be less than 50% w/w. The amount will range from about 100 to about 50% w/w for high molecular weight (≥500 kDa, preferably ≥100 kDa), multi-substituted polymers, such as hyaluronic acid, and from about 500 to about 50% c w/w for lower molecular weight polymers (≤100 kDa, preferably ≤50 kDa) such as PEGDA or PEGMAL.

Examples of reactive groups from different backbone chemistries that can be used to form a hydrogel with the appropriate crosslinkers are listed in the Table below. Some of the crosslinking reactions are initiated by UV light, while others are a chemical reaction.

TABLE 1
Reactive Groups and Crosslinking Chemistry

| Reactive Polymer | | Crosslink |
|---|---|---|
| (precursor) | Crosslinker | Mechanism |
| MeHA (methacrylated HA) | PEG diacrylate (PEGDA) | UV |
| | self-reactive | UV |
| AHA (acrylated HA) | dithiothreitol (DTT), PEG dithiol | chemical |
| | PEGDA | UV |
| | self-reactive | UV |
| ThHA (thiolated HA) | PEGDA, PEG divinyl sulfone, PEG | chemical |
| | dimaleimide | |
| PHA (pentenoate HA) | DTT, PEG dithiol | UV |
| NorHA (norbornene HA) | DTT, PEG dithiol | UV |
| PEGDA | self-reactive | UV |
| PEG diacrylamide | self-reactive | UV |
| 4 or 8-arm PEGMAL | DTT, PEG dithiol | chemical |
| | self-reactive | UV |
| 4 or 8-arm PEG vinyl sulfone | DTT, PEG dithiol, ethylene glycol bis- | chemical |
| | mercaptoacetate (BMA) | |
| | self-reactive | UV |
| 4 or 8-arm PEG acrylate | DTT, PEG dithiol, BMA | chemical |
| | self-reactive | UV |
| Collagen-SH (thiolated | PEGDA | Chemical |
| collagen) | | |
| Alginate | ion catalyst | Chemical |
| Polyvinyl alcohol acrylamide | self-reactive | UV |
| (PVA) | | |

Additional exemplary precursor compounds include, without limitation, non-alginate polysaccharides, collagen/gelatin, chitosan, agarose, and the like. These precursor compounds can be branched polymers with multiple arms or unbranched/linear polymer chains of a single backbone. They can be further functionalized for attaching dyes for visual observation. A particularly preferred hydrogel precursor compound is hyaluronic acid, or a hyaluronic acid/PEG mixture. The precursor compound(s) can also be functionalized with various chemical entities added to the precursor compounds at the time of hydrogel fabrication. These chemicals would be bound to the hydrogel matrix as opposed to being encapsulated in the interstitial voids for simple diffusion out of the microparticle.

Biocompatible hydrogels are particularly preferred. As used herein, “biocompatible” means that it is not harmful to living tissue, and more specifically that it is not biologically or otherwise undesirable, in that it can be administered to a subject without excessive toxicity, irritation, or allergic or immunogenic response, and does not cause any undesirable biological effects or interact in a deleterious manner. Biocompatible hydrogels would be selected to minimize any adverse side effects in the subject, as would be well known to one of skill in the art. Additional optional ingredients that may be included with the hydrogel precursor include fibronectin, laminin, collagen, other components of the extracellular matrix, and the like, including synthetic versions thereof.

The crosslinking profile of the microparticles can be tuned by adjusting the molecular weight of the precursor compounds, as well as the selected crosslinker, crosslinking conditions, and crosslinking process. For example, “tighter” or faster crosslinks may be desired in some embodiments to achieve a smoother bead surface and/or stronger gel by limiting the ability of molecules to leech out of the droplet/core into the surrounding environment during crosslinking. This can be achieved both by increasing molecular weight of the precursor species (e.g., ˜>40 kDa) and/or by decreasing the crosslinking time by adjusting the crosslinking chemistry or initiation method. Crosslinking can be carried out by various mechanisms depending upon the particular hydrogel precursor compound.

In general, one technique for preparing the microparticles is described in detail in U.S. Pat. No. 9,642,814, filed Jun. 3, 2015, incorporated by reference herein. The method involves preparing a hydrogel precursor solution consisting of the hydrogel precursor compound and a divalent cation (e.g., calcium, barium, strontium, and combinations thereof), dispersed or dissolved in a solvent system. The divalent cations are dispersed or dissolved in the solvent system along with the hydrogel precursor compound. The divalent cations should be included in the solution at a level of from about 0.025 moles/liter to about 0.25 moles/liter, based upon the total volume of the solution taken as 100%.

The hydrogel precursor solution can also include optional hydrogel crosslinking agents, catalysts, additives, media, nutrients, pH buffers, density modifying agents, viscosity modifying agents, or the like. The hydrogel precursor solution is then combined with alginate to initiate gelation of the alginate around the hydrogel precursor solution (via an “inside out” gelation process) to yield core/shell microparticles. Each core/shell microparticle comprises an alginate shell surrounding a liquid core, which comprises the hydrogel precursor solution. This generally involves dropwise addition of the precursor solution to an alginate bath, such as by generating/extruding droplets of the precursor solution that are dropped or sprayed into the alginate bath. The amount of alginate in the solution can be varied, but can range from about 0.1% to about 2.0% weight/volume, based upon the total volume of the solution taken as 100%. In general, the viscosity of the alginate solution should be less than the viscosity of the hydrogel precursor solution. The viscosity of an alginate solution depends upon the alginate concentration and average molecular weight of the alginate polymer (i.e., length of alginate molecules or number of monomer units in the chains), with longer chains resulting in higher viscosities at similar concentrations. In one or more embodiments, the viscosity of the alginate solution will range from about 1 to about 20 cP, and preferably from about 1 to about 4 cP at room temperature (˜20 to 25° C.). More specifically, the ratio of viscosity of the hydrogel precursor solution to the viscosity of the alginate solution should be greater than 1 at room temperature. In one more embodiments, the ratio of viscosity of the hydrogel precursor solution to the viscosity of the alginate solution is from about 1:1 to about 1000:1. In one or more embodiments, the ratio of viscosity of the hydrogel precursor is about 20:1. In one or more embodiments, the viscosity of the hydrogel precursor solution is from about 1 up to about 500 cP, with about 40 to about 100 cP at room temperature being particularly preferred. The pH of the alginate bath should range from about 6.2 to about 7.8 and preferably from about 6.6 to about 7.4.

The alginate shell forms from the inside out, and thickens around the droplets as the cations leach from the precursor solution droplet. In other words, the presence of the cation in the droplet causes alginate in the bath to agglomerate to the surface and crosslink around the droplet.

Next, gelation of the hydrogel precursor compound in the liquid core is initiated, such as by crosslinking and/or polymerization, to yield core/shell crosslinked microparticles. In one or more embodiments, the core/shell microparticles are combined with a hydrogel matrix crosslinker, preferably in solution. The crosslinker leaches through the alginate shell into the core/shell microparticles resulting in gelation (crosslinking) of the hydrogel precursor compound to form a 3-dimensional hydrogel matrix. The crosslinker will correspond to the hydrogel precursor compound, but can be varied to control the speed and level of crosslinking achieved within the resulting crosslinked matrix. In general, the crosslinker amount used in forming the microparticles will range from about 0.5 mM to about 30 mM depending upon the crosslinker and polymer system selected, preferably from about 1 mM to about 20 mM, more preferably from about 2 mM to about 15 mM, even more preferably from about 2.5 mM to about 10 mM, and even more preferably from about 2.5 mM to about 5 mM.

Each core/shell crosslinked microparticle comprises the alginate shell and a gelled core comprising a crosslinked, 3-dimensional hydrogel matrix. Crosslinking can be chemically induced, thermally induced, or photoinitiated, depending upon the particular precursor solution prepared. For example, the hydrogel crosslinker can be included in the alginate bath and diffuse through the alginate shell to crosslink the hydrogel microparticle in the core. Alternatively, the core/shell microparticles could be subjected to a source of UV radiation to initiate crosslinking in the hydrogel microparticle core. UV exposure times range from about 1 min. to about 10 min., preferably from about 2 min. to about 8 min., even more preferably from about 2.5 min. to about 5 min. The wavelength of the UV exposure will depend upon the photoinitiator and/or polymer system selected, but will generally range from about 100 nm to about 400 nm, preferably from about 315 nm to about 400 nm, and more preferably about 365 nm.

Suitable crosslinkers include photo- or thermal-initiated crosslinkers, chemical crosslinkers, such as acrylates, methacrylates, acrylamides, vinyl-sulfones, dithiols, and the like, which would be included as part of the alginate bath. Self-crosslinking hydrogel precursors could also be used. In these embodiments, a photoinitiator such as IRGACURE® 2959 (2-Hydroxy-4′-(2-hydroxyethoxy)-2-methylpropiophenone) or lithium phenyl-2,4,6-trimethylbenzoyl-phosphinate (LAP) may be included in the hydrogel precursor solution as the catalyst. The photoinitiator can also be included in the alginate bath. For chemical crosslinking systems, the crosslinking agent is typically provided in the alginate bath. For UV initiated crosslinking systems, the crosslinking agent can be included either in the alginate bath or in the hydrogel precursor solution along with the main polymer (backbone) species.

It will be appreciated that the precursor compounds are chemically altered in the final product via the crosslinking reaction to yield the fully crosslinked hydrogel matrix characterized as a porous elastic solid in defined particle form. For example, in one or more embodiments, the hydrogel matrix consists essentially (or even consists) of a crosslinked network of PEG and HA polymers containing thioether and ester crosslink bonds (e.g., for thiolated HA). However, it will be appreciated that the matrix may include trace amounts of unreacted precursor compounds, functional groups, and the like remaining in the network. Other examples of possible hydrogel matrices and crosslinks are described in the examples. The list provides examples, but is not exhaustive. Examples of backbone molecules that can be used include Chitosan, Agarose, Chondroitin Sulfate or a combination of molecules. Bonds formed between the backbones include chemical bonds with thioesther plus an ester, a thioesther plus an ester and a methyl on a beta carbon, a thioether sulfone, or a thioether succinimide. But other chemical bonds may be used. Photo (free radical) bonds could include ester plus ether or amide plus ether. It will be appreciated that there are increasingly complex combinations that can be utilized such that possible combinations are virtually limitless.

Once the core has been crosslinked, the alginate shell is then removed (e.g., with a chelating agent, and/or mechanical agitation, such as sonication) to yield the self-sustaining hydrogel microparticles or microbeads, having the characteristics described elsewhere herein. In other words, the alginate shell is not part of the final product and is always removed before use of the microparticles. The resulting hydrogel microparticles can be collected from the solution using a mesh screen or other device, and may be rinsed or suspended in medium, as desired.

Other fabrication methods include double emulsion, spraying, laser printing, 3D printing/additive manufacturing, and extrusion. Regardless of the fabrication method the resulting hydrogel matrix is characterized as being a semi-rigid network that is permeable to liquids and gases, but which exhibits no flow and retains its integrity in the steady state (i.e., a porous elastic solid). The hydrogel microparticle is a matrix-type capsule that holds the crosslinked fill material somewhat uniformly throughout the body of the bead, rather than having a distinct shell as in a core-shell type capsule. As noted above, each hydrogel microparticle is also a self-sustaining body having an outer surface and a defined/discrete 3-dimensional shape and geometry (as opposed to random particulates or aggregates). In other words, the microparticles of the present invention are not threads, fibers, or sheets, nor entangled threads, fibers, or sheets, nor agglomerates, nor aggregates, nor flecks, nor flakes, nor branched chain structures or polymer chains or branched fragments. In one or more embodiments, the resulting microparticles have a controlled size and are preferably substantially monodisperse with a coefficient of variation among a given batch of synthesized microparticles (i.e., the ratio of the standard deviation of the particles to the mean particle diameter) being 20% or less.

In one or more embodiments, the resulting microparticles are substantially spherical in shape (it being appreciated that the spherical body does not necessarily have to be perfectly round, but may be ellipsoidal, oblong, ovoid, teardrop, and the like). That is, the microparticle bodies are preferably substantially rounded and non-angular, preferably lacking of any sharp angles, with a substantially smooth outer surface, as exemplified in the images in the drawings. In one or more embodiments, the microparticles have a sphericity or “roundness index” of at least 0.25, preferably at least 0.35, more preferably at least 0.49, even more preferably at least 0.7, and even more preferably from about 0.75 to about 1. The sphericity or roundness index is the ratio of the surface area of a perfect sphere having the same volume as the given particle to the surface area of the given particle. The sphericity or roundness index can be calculated by

$$Ψ = \frac{{{π}^{\frac{1}{3}} ( 6 {V}_{p} )}^{\frac{2}{3}}}{{A}_{p}}$$

Where Vp is the volume of the particle, and Ap is the surface area of the particle (and a particle that is a perfect sphere would have a sphericity value of Ψ=1).

Advantageously, the particle size is highly customizable depending upon the capabilities of the selected droplet generator. In one or more embodiments, the resulting hydrogel microspheres or microparticles have an average (mean) particle size (i.e., average (mean) maximum cross-section surface-to-surface dimension) of greater than 30 μm, and in some case greater than 300 μm. For ease of reference, this cross-section dimension is referred to herein simply as the “size” of the microparticle. In one or more embodiments, the resulting hydrogel microspheres or microparticles have an average (mean) particle size of less than about 5 mm. Preferably, the resulting hydrogel microspheres or microparticles have an average (mean) particle size of less than about 2 mm, more preferably from about 30 μm to about 2 mm, even more preferably ranging from about 50 μm to about 1.5 mm, more preferably from about 150 μm to about 1.5 mm, even more preferably from about 300 μm to about 1.4 mm. In some cases, smaller microparticles ranging from about 30 μm to about 750 μm or 500 μm in size can be formed. As previously noted, regardless of the size, the substantially rounded particles have an aspect ratio of at least of at least 0.5, preferably at least 0.7, and preferably about 1.

In certain indications, it may be beneficial to produce a diverse range of sizes for a single application. In other situations, it may be beneficial for a product to have a uniform particle size among the particles in the suspension. In any event, the hydrogel microparticles of the invention are not nanosized and would not be considered nanoparticles or any other kind of nanocrystalline shape.

The durability of the microparticles can be adjusted by changing various parameters of the microparticle, including in the formulation and/or the processing parameters, such as polymer precursor mass fraction or molecular weight, crosslinker molecular weight, ratio of crosslinker and polymer precursor, crosslinker hydrolysis, crosslinking kinetics, crosslinking time, e.g., UV exposure time, and combinations thereof. Additional hydrogels are described in co-pending PCT/US2020/036361, filed Jun. 5, 2020, and incorporated by reference herein in its entirety.

The present microparticles are not drug delivery systems or vehicles for delivery of small molecule compounds, cells tissues, or other “therapeutics” as defined herein, and in most embodiments can be “empty”—that is, substantially free of such drugs, biologics, cells, tissues, or other therapeutic payload encapsulated therein or attached thereto. As used herein, a “therapeutic” is a compound or material having activity/bioactivity to treat or prevent a disease or condition that is not associated with or directly related to care and recovery of the site of placement of the soft tissue filler per se (for filling voids, plumping skin, smoothing wrinkles, etc.). Thus, the only active compounds that are contemplated are those intended to improve the outcome or recovery of the filler placement or implantation. The microparticles are also substantially free of metals, plastics, or ceramics. The term “substantially free,” as used herein, means that the ingredient is not intentionally added to the composition, although incidental impurities may occur, or residual/trace amounts may be left behind from the manufacturing process. In such embodiments, the hydrogel precursor solution compositions comprise less than about 0.05% by weight, preferably less than about 0.01%, and more preferably about 0% by weight of such an ingredient, based upon the total weight of the solution taken as 100% by weight.

Notwithstanding the foregoing, it is within the contemplated scope of the invention that non—“therapeutic” payloads could be co-delivered with the microparticles, such as a dye or other detectable label that can be used to visualize the microparticles. In one or more embodiments, a contemplated payload includes a local analgesic or anesthetic to reduce pain or infection at the injection site, such as lidocaine, antibiotics, anti-inflammatories, botox, or other active agents that directly contribute to success of the placement of the soft tissue filler, recovery, and outcome of the filler procedure, such as by reducing pain, bruising, or inflammation at the imputation site. Such analgesics or anesthetics are not otherwise intended to “treat” the patient beyond temporary pain relief, bruising, or inflammation, and as such are not included within the scope of “therapeutics.” The local analgesic, antibiotic, or anesthetic could be infused into the microparticle hydrogel matrix and/or may simply be mixed with the microparticles in suspension, e.g., as in the delivery vehicle, for administration of the composition.

Thus, compositions disclosed herein are placed as soft tissue fillers, such as into, above, or below the dermal, mid-dermal, subdermal, hypodermal, fascia, muscle, or periosteum regions of an individual by implantation (e.g., injection), for cosmetic or aesthetic procedures such as to plump thin lips, enhance shallow contours, soften facial creases, remove wrinkles, plump hands, and improve the appearance of scars, as well as for reconstructive surgery such as to fill voids due to loss or removal of tissue from surgery, trauma, etc. The route of administration of a hydrogel composition to an individual patient will typically be determined based on the cosmetic and/or clinical effect desired by the individual and/or physician and the body part or region being treated. A composition disclosed herein may be administered by any means known to persons of ordinary skill in the art including, without limitation, syringe with needle, a pistol (for example, a hydropneumatic-compression pistol), catheter, topically, or by direct surgical implantation or placement. The microparticles can be administered once or repeatedly, e.g., at regular intervals. Ultimately, the timing used will follow quality care standards. For example, microparticles disclosed herein can be administered once or over several sessions with the sessions spaced apart by weeks. As used herein, the term “dermal region” refers to the region of skin comprising the epidermal-dermal junction and the dermis including the superficial dermis (papillary region) and the deep dermis (reticular region). Thus, the microparticles disclosed herein can be administered to a dermal region of an individual by injection into, e.g., an epidermal-dermal junction region, a papillary region, a reticular region, or any combination thereof.

The compositions generally comprise a plurality of 3-dimensional hydrogel microparticles suspended in a pharmaceutically-acceptable delivery vehicle (typically at a physiologic pH ranging from 7 to 7.4 or slightly more alkaline, e.g., up to pH 8 or 9). The vehicle is preferably selected to be suitable for localized delivery (direct injection) at the site of implantation, such as through a small gauge needle. In one or more embodiments, the hydrogel microparticles may be lyophilized and stored as a dry powder before being reconstituted with suitable aqueous vehicle. In one or more embodiments, the hydrogel microparticles can be cryopreserved for storage and do not exhibit decrease in matrix quality upon thaw. Advantageously, due to the fully crosslinked nature of the microparticles, the soft tissue filler compositions described herein are heterogeneous mixtures or suspensions (not solutions) in which the microparticles are suspended in the delivery vehicle, preferably uniformly suspended, but remain and maintain as having their own distinct properties in the suspension. As such, the microparticles can be separated from the delivery vehicle (or other vehicle used for storage) through filtration if desired. Thus, the viscosity of the composition can be adjusted by adjusting the chosen delivery vehicle. In the case of aqueous delivery vehicles, including even sterile water, the compositions can be very thin and flowable with a low viscosity, or the microparticles can be suspended in a carrier vehicle having higher viscosity, such as a liquid polymer solution if desired. In either case, the fully crosslinked microparticles remain distinct components of the heterogenous composition, and can be filtered or separated therefrom if desired.

In one or more embodiments, delivery vehicles can be prepared as hyperosmotic solvent systems which reduce or minimize swelling of the microparticles while in suspension (and thus reduce their size). However, upon injection and exposure to fluids in the body (and change in salinity in the in vivo environment), the microparticles will swell and expand to their desired size to fill the defect void (e.g., wrinkle, etc.). In this manner the microparticles remain smaller until placed into the body, making injection through a small needle easier, while enabling expansion of the particles in the body to fill or expand in the place of implantation. Thus, the microparticles can be injected via any needle with preferable sizes of approximately 27-24 gauge or larger gauge needles even up to about 14 gauge depending on particle size and target site of implantation. In some embodiments, the microparticles may shear or fragment into smaller particulates upon passing through the needle.

In one or more embodiments, expansion and contraction of the microparticles (after initial formation) can be modulated by co-suspending the fully crosslinked microparticles in the carrier vehicle with a solute capable of inducing contraction of the microparticles as exemplified in the working examples. Increasing the concentration of this solute in the suspension causes the microparticles to contract. However, upon injection and exposure to fluids in the body (and change in concentration in the in vivo environment), the microparticles will swell and expand to their desired size to fill the defect void (e.g., wrinkle, etc.). In this manner the microparticle formulations remain smaller until in the body, making injection through a small needle easier, while enabling expansion in the body to fill a space.

In one or more embodiments, methods of dermal, cosmetic, or reconstructive filling are contemplated herein. The method generally comprises placing a plurality of hydrogel microparticles into or near a site of implantation in a subject, such as by injection or implantation. These novel soft tissue fillers are useful for treating skin conditions, typically the loss of volume in the face and extremities and the filling of wrinkles. The implanted hydrogel microparticles provide cushioning volume and fill at the site of implantation. As demonstrated in the working examples, the microparticles also induce a favorable physiological response in the subject at the site of implantation. For example, the microparticles do not induce unwanted inflammatory responses at the site of implantation. In one or more embodiments, methods contemplated here involve reducing wrinkles, adding volume and aesthetically correcting surgical or injury-related deformities from the dermal layers down to the periosteal layers such as following severe burns, tumor, or tissue removal (e.g., lipoma removal). The microparticles help restore or maintain a structure and cushion within the skin. Moreover, due to their elastic nature, they demonstrate an elastic response that mimics the native tissue.

The methods can be applied in a variety of human applications including, but not limited to, wrinkles in the face, neck and hands and volume filling in the same sites along with the arms, legs, abdomen, hands, and feet. Preferably, the microparticles or composition comprising the microparticles is introduced into the dermis region as well as subdermally in the subcutaneous tissue or even to the periosteum.

For treatment methods, the microparticles are suspended or dispersed in a suitable delivery vehicle for administration to the subject. Exemplary delivery vehicles will include biocompatible liquid suspensions, viscous solutions, putties, pastes, or gels in which the microparticles are distributed, such as compatible biologic fluids, and uncrosslinked or partially crosslinked, low concentration HA liquid solutions. Saline solutions or other buffered solutions may also be used as vehicles for delivery. The injected composition could contain a single formulation of microparticles within a narrow size range (substantially monodispersed), for example 400-600 μm diameters. Conversely, the composition could be a mixture of formulations having different microparticle sizes (polydisperse). For example, it may be advantageous to mix different formulations of hydrogels to have a combination of microparticles that degrade at different time points, or have different stiffness characteristics. Thus, composition of different microparticles are contemplated herein, comprising a first population of microparticles having a first characteristic (e.g., size range, stiffness, and/or degradation profile, etc.) and a second population of microparticles having a second characteristic (e.g., size range, stiffness, and/or degradation profile, etc.), where the second characteristic is different from the first characteristic. The mixed compositions can include 2, 3, 4, 5, 6, 7, 8, 9, or 10 different microparticle formulations or more. Likewise, the viscoelastic microparticles of the invention may be mixed with viscous HA that is not crosslinked or only partially crosslinked or other viscous dermal fillers (as the carrier vehicle) yield a suitable formulation. The product could, for example, be composed of a 1:10 ratio of microparticles suspended in a viscous dermal filler such as partially crosslinked HA.

More generally, the methods comprise (or consist of) locally administering a cosmetically or aesthetically effective amount of the resulting microparticle composition to the patient. Administration generally includes direct injection of the microparticle composition at or near the target site. Advantageously, the durable microparticles are generally confined to the localized region for a cosmetically or aesthetically effective period of time. As used herein, the term “cosmetically or aesthetically effective” refers to the amount and/or time period that will maintain the cosmetic or aesthetic response of a tissue, sought by a researcher, clinician, or patient.

The treatment may be repeated via additional injections or infusions if necessary. Those skilled in the art will appreciate that treatment protocols can be varied depending upon the particular aesthetic target and preference of the medical practitioner or researcher. Examples of typical injection procedures include serial injections, linear threading, fanning injections and cross-hatching for larger surface areas. The compositions described herein are contemplated particularly for use as dermal fillers.

Advantageously, the hydrogel microparticles are durable, meaning they will not break down or degrade under storage conditions for at least 6 months in PBS at 37° C. In other words, the durable microparticles have “in vitro storage stability” of greater than 6 months. In one or more embodiment, durable hydrogel particles are shelf-stable in PBS at room temperature (27° C.) for one year or more, with current data exemplifying microparticles with a shelf-life of more than 4 years. Preferably, when such durable hydrogel microparticles are implanted, they will not break down for at least 3 months, and more preferably for at least 6 months under normal physiological (aka normal in vivo clearance of foreign bodies via phagocytosis, degradation, adsorption, etc.) conditions.

A particular advantage of the inventive compositions and microparticles is their improved “stickiness” as compared to other types of microparticles when implanted. That is, the microparticles have a tendency to adhere or cling to the tissue in vivo at the site of implantation and shown in the photographs in the examples below. This further enhances the effectiveness of the treatment by maintaining the localized filling at the site of implantation. Further, the microparticles demonstrate no induction of any inflammatory response at the site of implantation, and/or any foreign body response, including lymphocytes or collagen ring formation.

Another particular advantage of the inventive compositions and microparticles is their ability to be degraded on demand. This is important for situations where the end-user may incorrectly apply the product to a specific region, the cosmetic effect is not as desired, or in situations where an unanticipated effect, such as a Tyndall effect, may be noted. At that time, the hydrogel microparticles would need to be dissolved within hours to days. A number of different approaches can be taken to achieve dissolution on demand, such as described in the examples below and summarized in Table 5 and Table 6.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### Examples

The following examples set forth methods in accordance with the invention. It is to be understood, however, that these examples are provided by way of illustration and nothing therein should be taken as a limitation upon the overall scope of the invention.

### Characteristics of the Final Hydrogel Microparticles

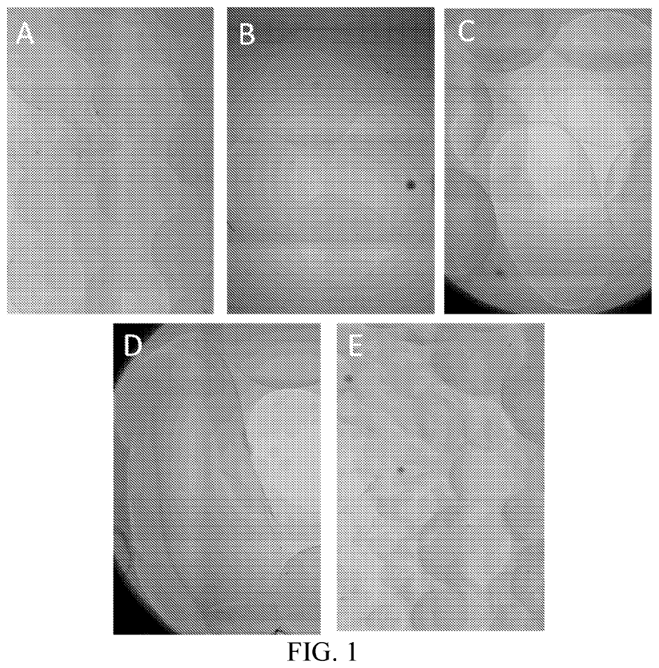

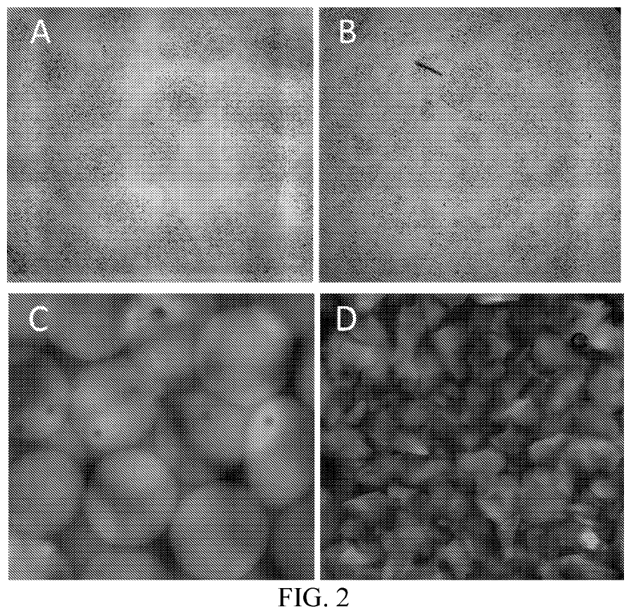

Crosslinked microparticles can be constructed using a number of different manufacturing methods including (but not limited to): core shell spherification, emulsion, extrusion of alginate, patterned molds, and printed. Microparticles will typically be small enough to be injected through a needle or infused through a catheter. We have manufactured microparticles in the size ranges of under 150 μm to over 1,500 μm, so that they can pass through 18 to 30 G needles, which makes them easily injected into the skin. However, the microparticles can be made with diameters of <50 μm so that even smaller needles can be used. The size and shape of the microparticles can be altered to meet the therapeutic goal. FIG. 1 illustrates examples of microparticles as spheres (FIG. 1A), teardrops (FIG. 1B), ovals (FIG. 1C), and organic large tube shapes (FIG. 1D). The product could also be shaped as tubes, rectangles or other shapes. In addition, a mixture of shapes could be manufactured and used in the same product as shown in FIG. 1E. The particles can withstand physical forces such as shearing. However, when they are put under excessive force they break up into smaller pieces but maintain a distinct physical shape. This is different from paste-like lubricant gels, which do not maintain a physical border and distinct shape. FIG. 2 shows MeHA microparticles prior to exposure to shear forces (FIG. 2A) and after (FIG. 2B). While the shear forces can break apart the microparticles, they still maintain the solid-like form. AHA microparticles impregnated with a fluorescent probe are shown prior to exposure to shear forces (FIG. 2C) and after (FIG. 2D).

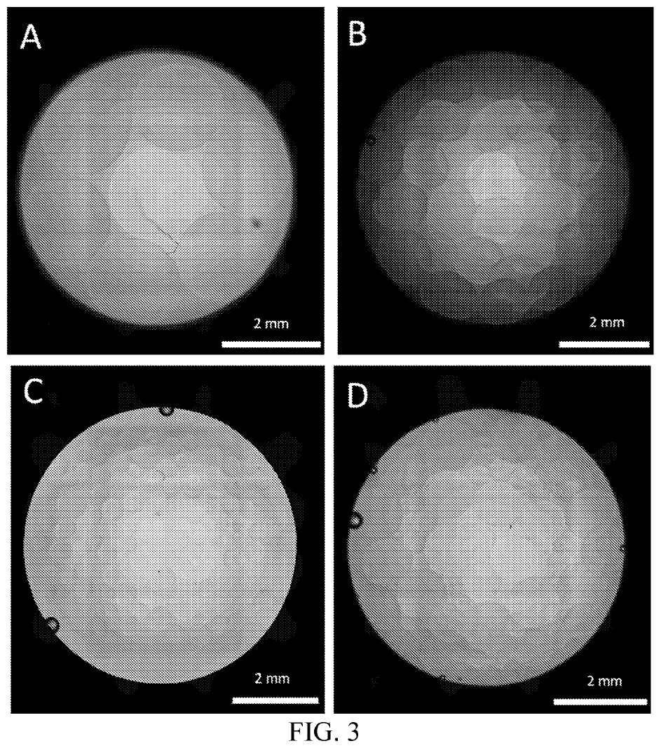

Likewise, the microparticles can be created in a wide range of sizes from an average of 100-2000 microns as shown in FIG. 3. Large microparticles are shown in FIG. 3A, medium size in FIG. 3B, small microparticles in FIG. 3C and a mixture of sizes in FIG. 3D. In theory, the mixed large and small sizes may provide a smoother final product as a dermal filler.

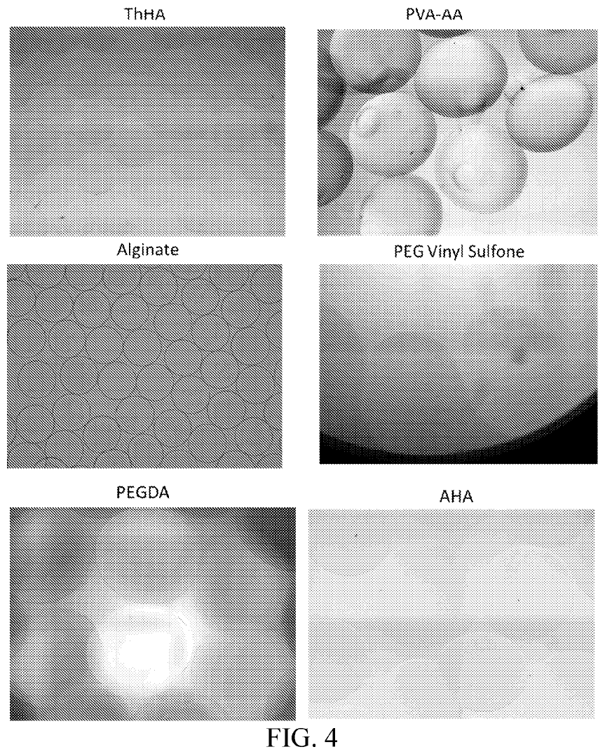

By utilizing different precursor polymers and crosslinking schemes the crosslinked microparticles can be manufactured from a variety of materials. FIG. 4 provides a few examples of microparticles made from thiolated hyaluronic acid (ThHA), polyvinyl alcohol acrylamide (PVA-AA), diacrylate polyethylene glycol polyethylene diacrylate (PEGDA), alginate, polyethylene vinyl sulfone, and acrylated hyaluronic acid (AHA).

The hydrogel microparticles can advantageously be fabricated from a variety of slow-gelling polymer precursors, such as hyaluronic acid (HA) and polyethylene glycol diacrylate (PEGDA), polyethylene glycol maleimide (PEGMAL), multi-arm PEGs, hyaluronan, fibrin, chitosan, collagen, heparin, polylactic acid (PLA), poly(L-lactic acid) (PLLA), polylactic-co-glycolic acid (PLGA), polycaprolactone (PCL), polyvinyl alcohol (PVA), polyvinylpyrrolidone (PVP), methacrylic acid (MAA), 2-hydroxyethyl methacrylate (HEMA), polyacrylamide (PAM) extracellular matrix, and functionalized varieties of the same (e.g., acrylated, methacrylated, thiolated, etc.) or multi-arm varieties of the same (e.g., 4-arm PEG, 8-arm PEG). However, any crosslinkable hydrogel precursor compounds would be suitable for use with the invention, with preferred compounds being biocompatible homopolymers or copolymers, and particularly block copolymers, as well as other types of crosslinkable monomers and/or oligomers.

Further characterization of some of the chemical version of the end products are provided in Table 2. The precursor polymer and potential crosslinkers along with the crosslinking mechanism are shown. The first row indicates the variety in viscoelastic solid-like microparticle characteristics including the reactive groups, crosslinkers, crosslinking time, range of polymer mass and the range of particle diameters. Different crosslinking mechanisms can be utilized including photo-initiation and chemical reactions (Table 2) and as such the different crosslinking approaches require different amounts of time to cure as shown in Table 2.

TABLE 2
Viscoelastic Solid-Like Microparticle Formulations

| | | | | | Range of | |
|---|---|---|---|---|---|---|
| | | | | | Polymer mass | Range of |
| | | | | | % in | Sphere |
| Base | Reactive | | Cross linking | Crosslinking | Crosslinked | Diameters |
| Polymer | Group | Crosslinker(s) | Mechanism | Time | Gels | (microns) |
| Polyethylene | Acrylate | Self-reactive | Free radical | 30-120 | 1.0%-5.0%* | 300-2500 |
| glycol | | | photopolymerization | seconds | | |
| (PEG) | Vinyl | DTT, PEG-dithiol, | Michael | 30-60 | 0.75%-3.0% | 800-2500 |
| | Sulfone | multi-arm PEG-thiol, | addition | minutes | | |
| | | ethylene glycol bis- | | | | |
| | | mercaptoacetate | | | | |
| | Maleimide | DTT, PEG-dithiol, | Michael | 5-60 | 0.5%-5.0% | 600-2500 |
| | | multi-arm PEG-thiol | addition | minutes | | |
| Hyaluronic | Acrylate | Self-reactive | Free radical | 120-300 | 0.25%-1.5% | 250-2000 |
| Acid | | | photopolymerization | seconds | | |
| | Methacrylate | Self-reactive | Free radical | 90-120 | 0.25%-1.5% | 250-2000 |
| | | | photopolymerization | seconds | | |
| | Thiol | PEG-diacrylate, multi- | Michael | 5-60 | 0.5%-3.5%* | 600-2000 |
| | | arm PEG-acrylate, PEG- | addition | minutes | | |
| | | dimaleimide, multi-arm | | | | |
| | | PEG-maleimide | | | | |
| Polyvinyl | Acrylamide | Self-reactive | Free radical | 120 | 1.0-5.0% | 400-2000 |
| alcohol | | | photopolymerization | seconds | | |
| (PVA) | | | | | | |
| Sodium | Guluronic | Divalent cations | Ion chelation | Instantaneous | 1%-2% | 150-2000 |
| Alginate | Acid | (calcium, barium, | | | | |
| | | strontium) | | | | |

The microparticles listed in Table 2 have vastly different swelling characteristics. For example, AHA swelling ratio (Q value) is approximately 250 while ThHA is only 28. The hydrogel chemistry provides a high level of control over the swelling characteristics such that it is possible to maintain the microparticles in an environment that inhibits swelling until after injection. Such a situation would be advantageous because a smaller needle and entry point in the skin could be used and still provide high degrees of increased volume.

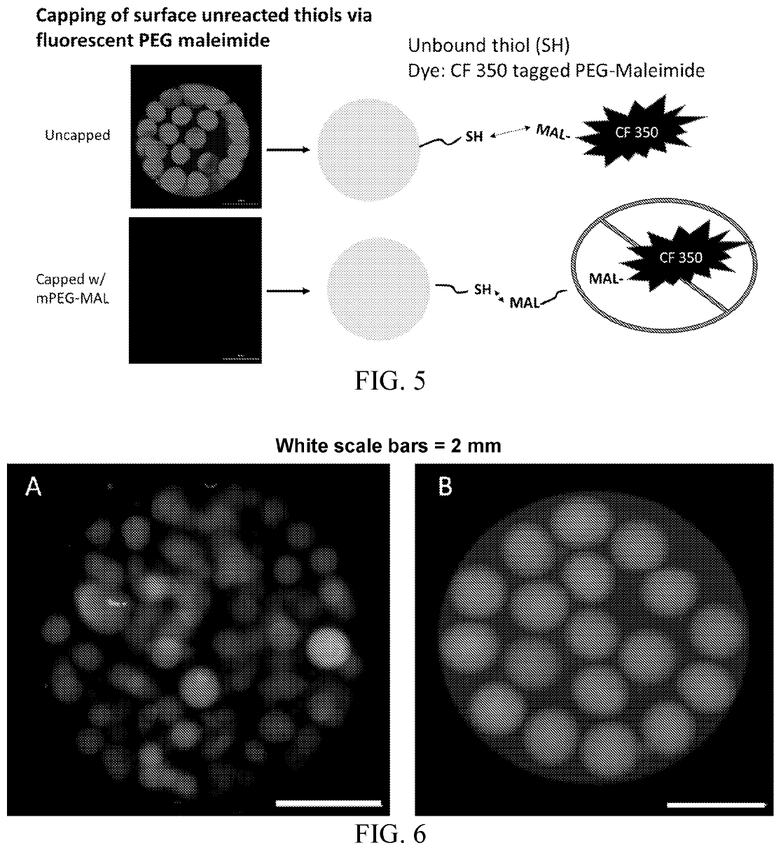

In addition to different crosslinking approaches, additional chemical modifications can be made to alter the characteristics of the microparticles. For example, unreacted species within the fully-crosslinked particle can be further modified as shown in FIG. 5. This can be done to alter the interaction of the particle with the surrounding tissue either making it less reactive or more reactive, depending on the application. In this example, low levels of residual reactive groups are commonly present within a hydrogel after crosslinking. These groups can be utilized, if desired, for downstream functionalization. In this example, a monofunctional PEG-maleimide “molecular cap” is used to quench residual thiol groups. As seen in the example, treatment of the hydrogel spheres with the molecular cap successfully blocked binding of a maleimide conjugated fluorescent dye, in contrast to untreated spheres which readily bound the dye.

The approaches described provide a wide range of options for producing viscoelastic solid-like dermal fillers. Contrasting the properties of the viscoeleastic microparticles described here with the currently marketed paste-like dermal filler, reveals the differences. First, in the cosmetic field it is known that the higher the molecular weight of the filler, the longer the duration in vivo. Table 3 compares the molecular weight of the final crosslinked solid-like microparticles to the currently marketed paste-like fillers. The starting molecular weight of the precursor to the solid-like viscoelastic microparticles can be 0.2 MDa as shown in the example in Table 3, but when fully crosslinked the value increases to the point that it is undeterminable. Basically, the entire bead acts as a single molecule. In contrast the currently marketed paste-like dermal fillers may start with a higher molecular weight precursor, but studies have shown that the actual final product has a lower molecular weight after processing.

The percentage of crosslinking within the product is also vastly different for the viscoelastic solid-like microparticles compared to the paste-like fillers. The viscoelastic solid-like microparticles once formed, preferably have 20% or greater crosslinking, preferably 20%-30% crosslinking, while the other products are all below 20% (Table 3). The particle sizes are all in similar ranges however the viscoelastic solid-like microparticles have a defined non-angular surface. The final concentration of HA in the products is nearly twice as high in the viscoelastic solid-like microparticles compared to the paste-like products. The durability of paste-like fillers are directly related to the molecular weight of the precursors and the crosslinking of the gels. Table 3 shows that the molecular weight of the finished product is not determined because the crosslinking results in a single microparticle with a MW that is beyond measurement. It is important that stiffness is not high, as it could result in a less natural appearance. Thus, the low stiffness (G′) value compared to the currently marketed products is advantageous. It should also be noted that stiffness measurements on microparticles are different than on bulk gels and thus the numbers cannot be directly compared.

TABLE 3
Viscobead Comparison with Current HA Cosmetic Fillers

| | Precursor | Finished | | Particle | HA | Stiffness |
|---|---|---|---|---|---|---|
| | MW | Product MW | Crosslinking | Size | concentration | G′ |
| | (MDa) | (MDa) | (%) | (μm) | (mg/mL) | (Pa) |
| Viscoelastic | 0.2 | Indeterminable | 20-30% | 350-1000 | 40 | 3.45 |
| solid-like | | | | | | |
| microparticle | | | | | | |
| RESTYLANE ® | 1.5-2.0 | 0.49 | <1 | 250-300 | 20 | 175 |
| PREVELLE ® | 1.5-2.0 | 1.5-2.0 | 12 | 350 | 5.5 | 1800 |
| JUVEDERM ® | 2.5 | 0.65 | 6 | 703 | 24 | 76-111 |
| ULTRA | | | | | | |
| HYLAFORM ® | 4.0-6.0 | 4.0-6.0 | 12-20 | 500 | 5.5 | 100 |

The same general differences can be applied when comparing a viscoelastic solid-like hydrogel microparticle with non-HA paste-like products currently marketed (Table 4). The initial and final molecular weight of the non-viscoelastic pastes likely do not change, although no independent measurements have been reported. However, the viscoelastic solid-like microparticle has a dramatic increase in the molecular weight as described for the microparticles based on HA. The differences again result in a stiffer, and assumed, more durable product.

TABLE 4
Viscobead Comparison with Current Non-HA Cosmetic Fillers

| | Viscoelastic | | | |
|---|---|---|---|---|
| Filler/ | solid-like | | | |
| Property | microparticle | RADIESSE ® | SCULPTRA ® | ARTEFILL ® |
| Microsphere | Polyethylene | Calcium | Polylactic acid | Poly(methyl |
| chemistry | diacrylate | hydroxyapatite | (PLA) | methacrylate) |
| | (PEGDA) | (CaH) | | (PMMA) |
| Precursor | 0.0034 | 0.000502 | 0.140 | NR |
| Molecular | | | | |
| weight (MDa) | | | | |
| Final Product | Indeterminable | 0.000502 | 0.140 | NR |
| Molecular | | | | |
| Weight (MDa) | | | | |
| Particle Size | 350-1000 | 25-45 | 40-65 | 30-50 |
| (mm) | | | | |
| Concentration | 300 | 300 | 8 | 200 |
| (mg/mL) | | | | |
| Stiffness (Pa) | NR | 1,407 | 977 | NR |

NR = Not Reported

### In Vitro Degradation Rate

In contrast to these uncrosslinked forms of HA, one formulation of our fully-crosslinked HA microparticles have an in vitro half-life of approximately 50 days while microparticles made of a formulation based on polyethylene glycol have an indefinite duration in vitro. In this experiment microparticles were maintained at 37° C. and 10% humidity for the 50 days.

In the body, the degradation of the HA microparticles is dependent on endogenous enzymes such as hyaluronidase. In vitro studies in the preliminary stages will demonstrate the enzymatic degradation of the fully-crosslinked microparticles. To be able to monitor the microparticle degradation in vivo, we developed fluorescent microparticles. SAMSA Fluorescein was added to the precursor AHA solution shown in FIG. 6A. FIG. 6B shows fluorescent labeling of PEGDA microparticles.

### Solute-Controlled Microparticle Size

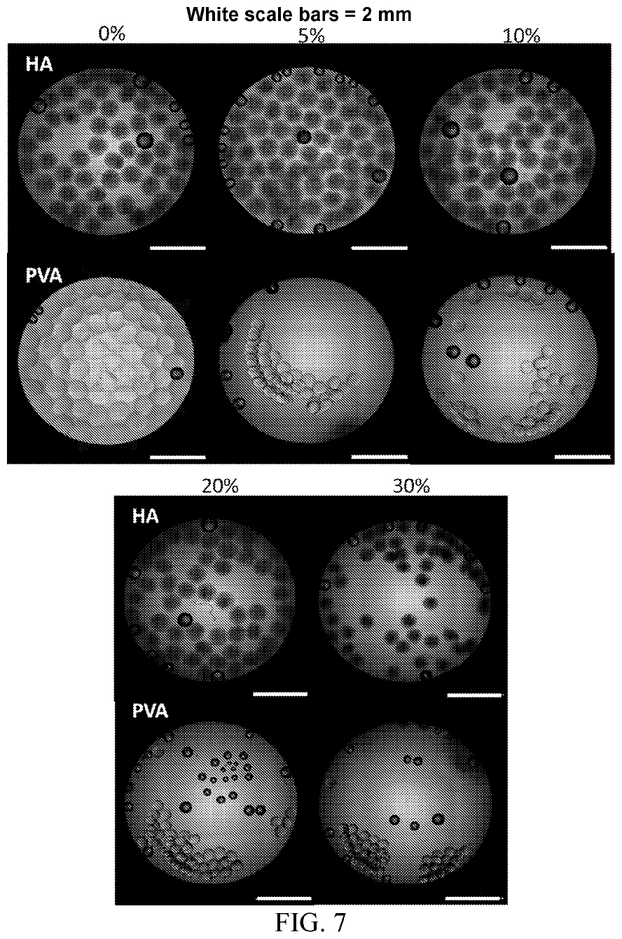

Changes in the microparticle diameter can be obtained through a solute-controlled approach, which utilizes heuristics of a process known as aqueous two-phase extraction in the context of hydrogels (Gehrke et al., 1998). This approach has been utilized in the past to load hydrogels with proteins. In this iteration, it is used to reduce the size of the microparticles for ease of injection with through small needles. The hydrogel microparticles are typically stored in a normal phosphate buffered saline solution. However, when a polymer such as PEG is co-dissolved in the storage solution, a second aqueous phase is formed within the hydrogel network This ultimately disrupts the interaction of water with the hydrogel polymer, which reduces the swelling force and therefore the size of the microparticle. For the experiments described in FIGS. 7-11, PEG (MW 20 kDa) was dissolved at 5, 10, 20, and 30% by mass in the storage solution. Reduction in the size was directly related to the concentration of PEG in solution, as shown in FIG. 7. In the figure, HA-based microbeads illustrated minimal diameter reduction in response to increasing solute concentrations while a PVA-based hydrogel shows a dramatic reduction in diameter in response to the same solute concentrations.

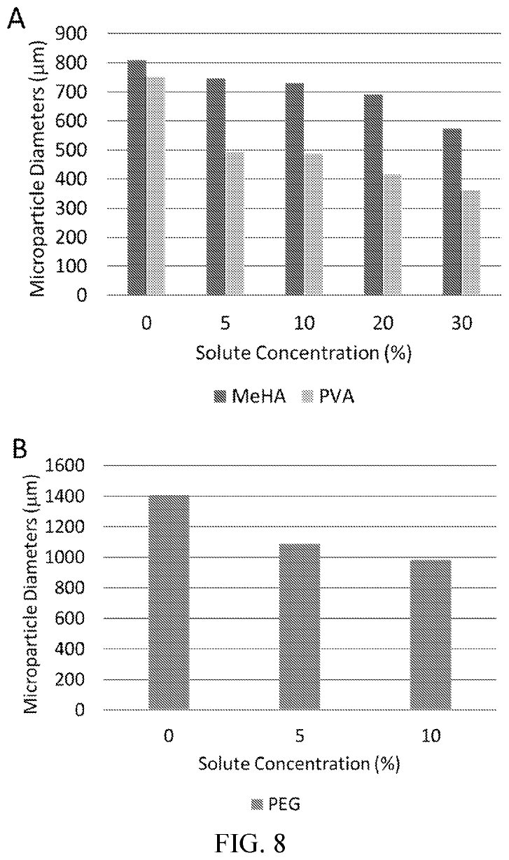

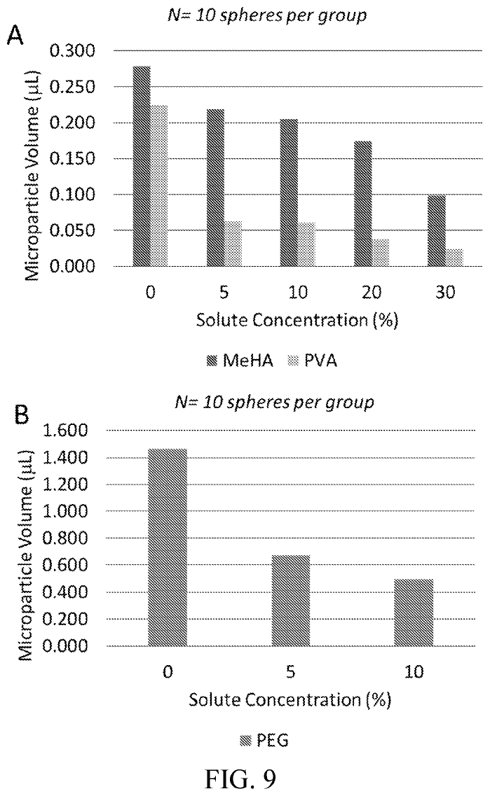

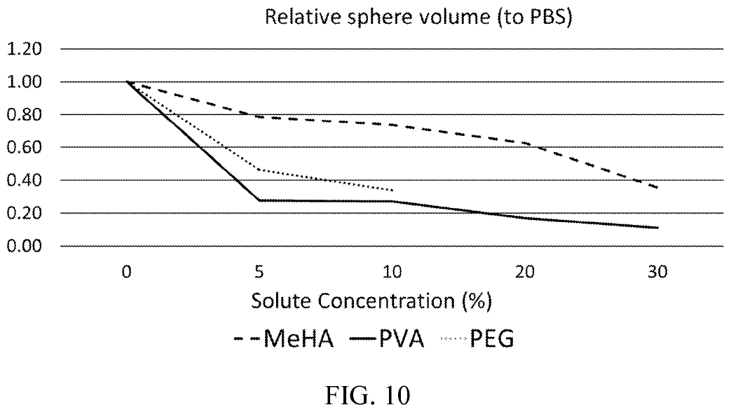

FIG. 8 provides comparisons of different hydrogel formulations and their ability to reduce in size. FIG. 8A is a comparison between HA hydrogel microparticles and PVA hydrogel microparticles. Both decreased in diameter with increasing percentages of solute up to 30%. In FIG. 8B the lower solute concentrations also induced a decrease in diameter. At the highest concentrations of the solute, the microparticles became impossible to image. The same trends were noted when the volume of microparticles were measured (FIGS. 9A and B). The changes in volume with solute concentrations are tracked visually in FIG. 10.

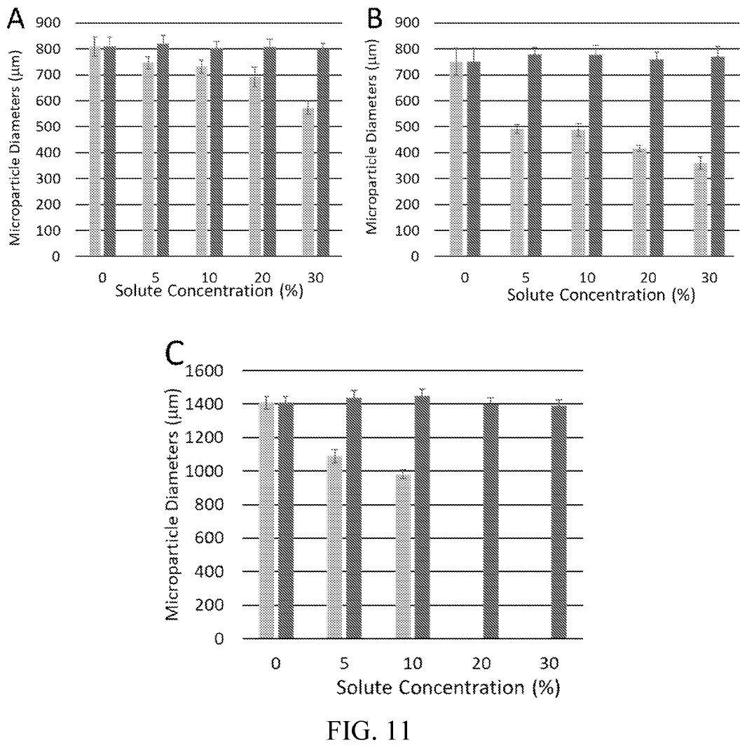

The reduction in diameter is reversible by exchanging the solute-containing storage solution with normal phosphate buffered saline. FIG. 11A shows the level of reduction in the diameters of the microparticles with increasing solvent concentrations (light gray). When the microparticles were returned to solutions without solute, the diameter of the microparticles returned to their original size. FIG. 11A shows the changes in diameter of HA-based microparticles. FIG. 11B shows the changes in PVA microparticles. FIG. 11C illustrates the change in diameter of PEG-based microparticles. The change in size of the PEG microparticles could not be measured while in high concentrations of the solute but the microparticles could be visualized when returned to solute-free conditions (dark gray).

### Biocompatibility of Microparticles

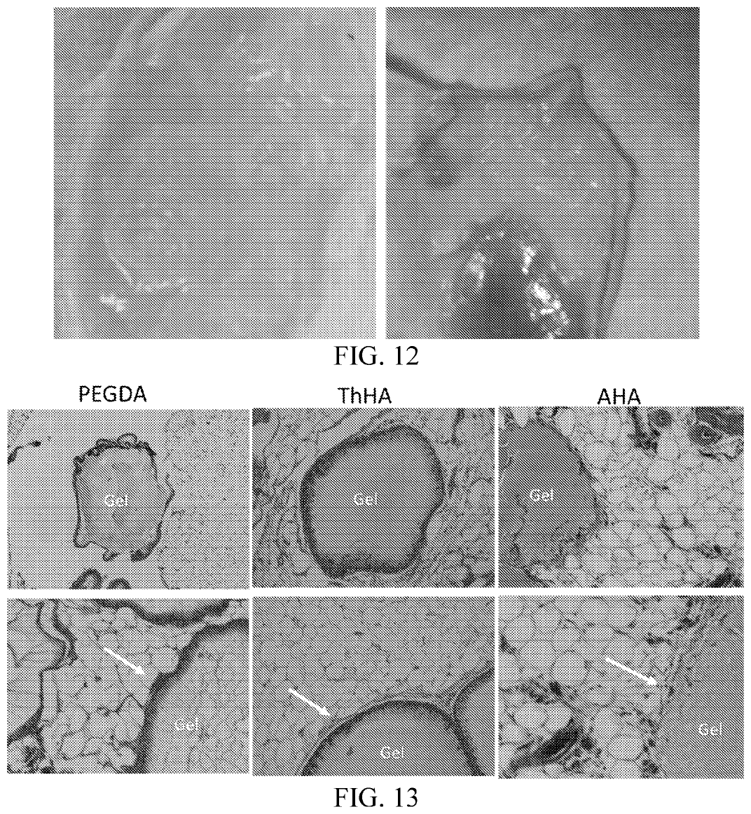

Microparticles were injected subcutaneously in rats to test for biocompatibility. The microparticles were injected in either a bolus amount (FIG. 12A) or injected as individual microparticles (FIG. 12B). The images were captured 2 weeks after the microparticles had been injected and there were no signs of gross inflammation (redness or swelling). The biocompatibility of microparticles is exemplified in FIG. 13 where hydrogel particles made of PEGDA, ThHA, or AHA, using the same technology. The microparticles were injected intraperitoneally into rats and after 2-10 weeks, intact hydrogel microspheres were still found in the region. Areas of fibrosis (collagen ring) and inflammatory response (macrophages and lymphocytes) were measured at 4 different locations around each microsphere by 2 blinded technicians. Only limited fibrosis was found surrounding some of the microparticles (FIG. 13; ThHA). The AHA microparticles had the lowest percentage of fibrosis surrounding them. When fibrosis was found surrounding the AHA microparticles, it was much smaller than particles made of other components. In addition, we measured the width of fields of inflammatory cells surrounding the microparticles. Fewer AHA samples showed signs of inflammatory cells surrounding the microparticles.

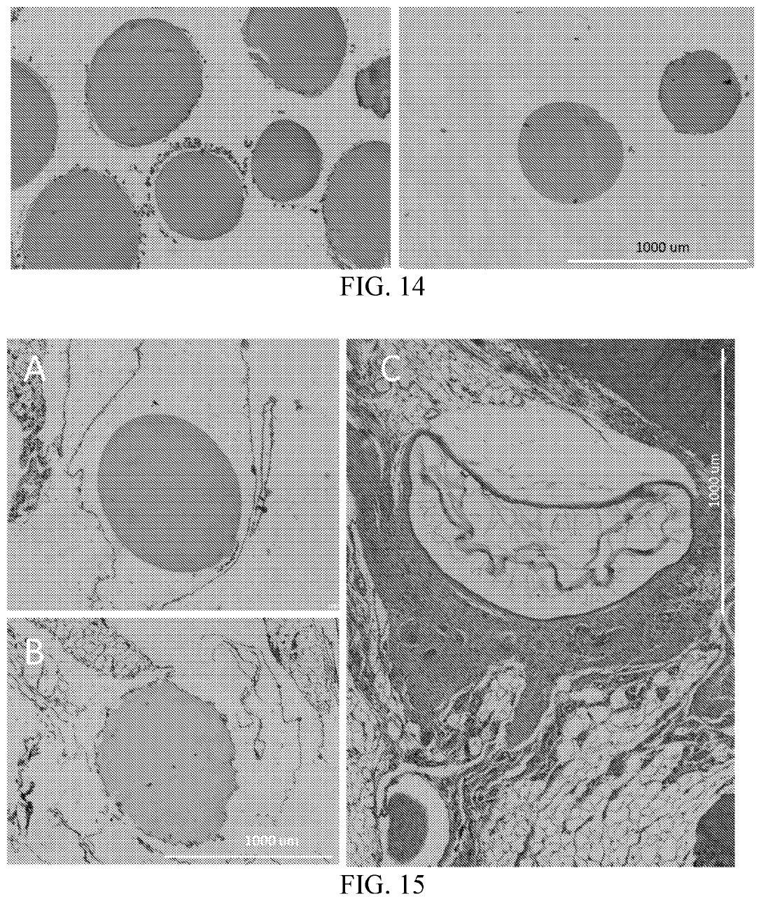

Not only is there little or no response in the surrounding tissue, when loose viscoelastic microparticles are removed from the site, there are no inflammatory cells or fibrosis covering the biocompatible materials, as shown in FIG. 14. The image on the left shows a few inflammatory cells circling or attached to the microparticles, but no ring of fibrosis present.

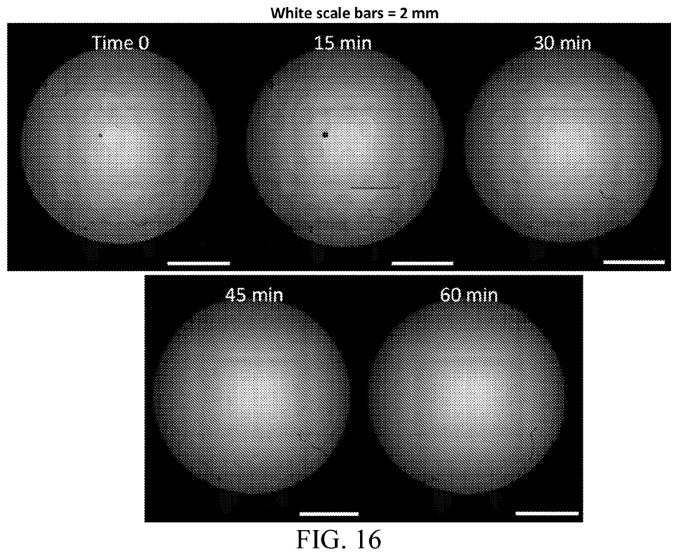

As the solid-like microparticles dissolve or otherwise degrade, the degradation starts from the surface and still does not elicit a foreign body response (FIG. 15). FIG. 15A shows a newly deposited AHA microparticle with no foreign body response in the surrounding omentum. Ten weeks after implantation, the microparticle is degrading from the surface and still elicits no foreign body response (FIG. 15B). This is in contrast with non-biocompatible materials, which can elicit a strong foreign body response that destroys the microparticle as shown with the example in the rat omentum in FIG. 15C.

### Designed Degradation

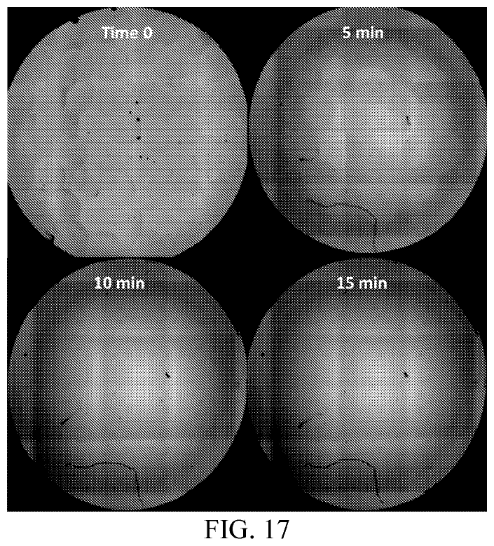

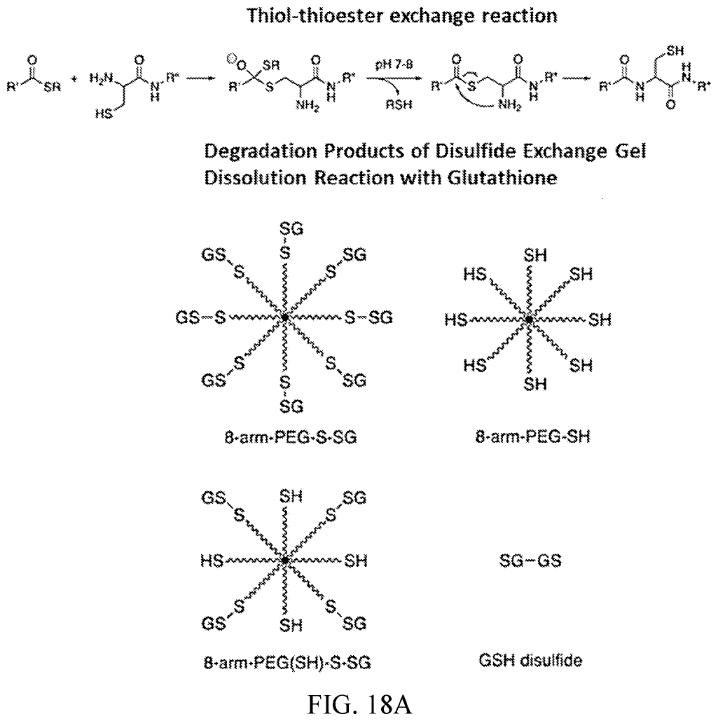

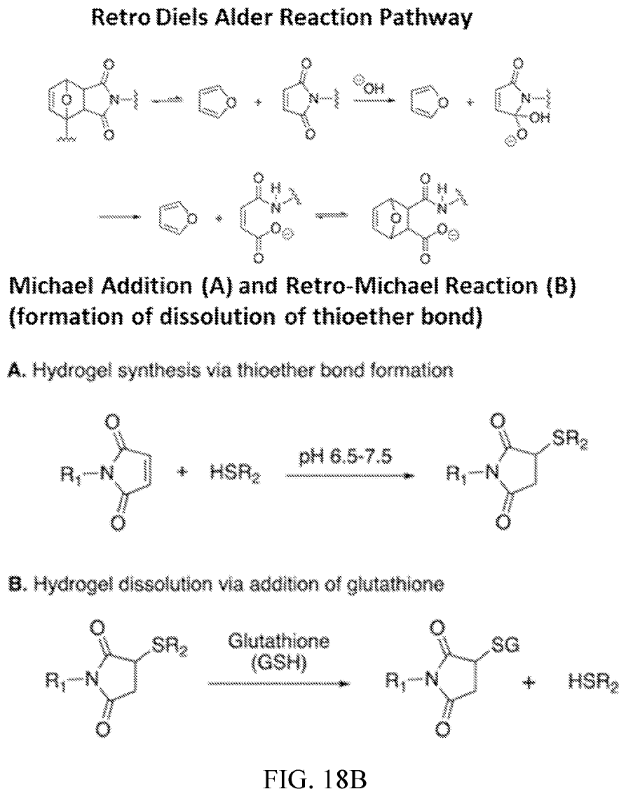

Occasionally, based on user error or the physiology of the recipient, such as allergies, the microparticles may have to be removed. The crosslinking chemistries can be designed so that the beads break apart in hours to days. FIG. 16 shows the degradation of microparticles secondary to exposure to an enzyme such as hyaluronidase, in the case of HA-based microparticles. An alternative method of designed degradation utilizes chemical dissolution. In FIG. 17, microparticles were exposed to DTT and were completely dissolved in 15 minutes. FIGS. 18A-B illustrate some of the chemistries behind the designed degradation describing the resulting byproducts.

If chemically crosslinked microparticles are involved, the dissolution reagents could be injected into the area of concern. Table 5 outlines the possible crosslink reactions and the dissolution reagent that would be used in each situation. Reagents such as glutathione could be injected into the site of hydrogel microparticles containing disulfide bonds for on demand dissolution.

TABLE 5
Chemically Crosslinked Hydrogel Dissolution on Demand

| | | | Example |
|---|---|---|---|
| Dissolution | Stability and | Example Hydrogel | Dissolution |
| Mechanism | Degradation Properties | Systems | Reagents |
| Disulfide Exchange | Less stable hydrogel; fast | 1. R-SH + R-S-TP | 1. L-cysteine |
| | degradation (minutes to | (sulfur-thiopyridine); | 2. glutathione |
| | hours) | 2. R-SH + Hydrogen | 3. cysteine methyl |
| | | Peroxide | ester |
| Thioester Exchange | Moderately stable | 1. R-SH + R- | 1. L-cysteine |
| | hydrogel; fast degradation | succinimide (SVA, | 2. glutathione |
| | (minutes to hours) | NHS, etc) | 3. cysteine methyl |
| | | 2. general crosslinker | ester |
| | | with incorporated | |
| | | thioester group | |
| Retro-Michael | Stable hydrogel, medium | 1. R-SH + R- | 1. glutathione |
| reaction (thioether | to slow degradation (hours | Maleimide | |
| dissolution) | to days) depends on | 2. R-Norbornene + R- | |
| | several factors) | SH + UV | |
| Retro Diels-Alder | Stable hydrogel; slow | 1. R-Furan + R- | High temperature |
| reaction | degradation (multiple days, | Maleimide | (>60 C.) |
| (cycloaddition- | affected by temperature) | | |
| fragmentation) | | | |

In addition to chemical dissolution of the viscoelastic hydrogel microparticles, physical modalities can be used to break the crosslinking bonds and dissolve the microparticles. Table 6 provides some examples of the hydrogel microparticle chemistry and possible methods of physical and chemical dissolution. For example, hyaluronic acid microparticles crosslinked with methacrylates could be dissolved using an enzyme such as hyaluronidase or by excitation light emitted from at wo-photon laser. In another example alginate microparticles could be dissolved by applying high intensity ultrasound to the surface of the skin. Visible and UV light can also be used to dissolve microparticles based on Polyvinyl alcohol, crosslinked with thiols. Red light could be used to dissolve microparticles based on poly(acrylic) acid crosslinked with Azo groups. With these methods, handheld devices could be applied to the skin to dissolve the viscoelastic solid-like microparticles. In contrast, some of the current dermal fillers have no method of dissolution. Such is the case for ARTEFILLL® made of PMMA (Table 4). If this product must be removed, the only option is invasive surgery.

TABLE 6
Physical Dissolution of Microparticles

| | Crosslinking chemistry, Initiator, | |
|---|---|---|
| Polymer | or Gelation mechanism | Dissolution Technique |
| Hyaluronic acid | Methacrylates, thiol, aldehyde | Two-photon laser |
| | Adamantan and beta-cyclodextran | Ultrasound |
| | Methacrylates, 4-vinylbenzl | Two-photon laser |
| | chloride | |
| Collagen | Thiol and 3,5-dichloro-1,2,4,5 | UV and Visible light |
| | tetrazine | |
| Gelatin | Methacrylates, thiols | Two-photon laser |
| Alginate | Ca²⁺, Ba²⁺, Mg²⁺ | Two-photon laser |
| | Cysteine groups | High-intensity frequency |
| | | ultrasound |
| Polyethylene glycol | Methacrylates, maleimides, vinyl | Two-photon laser |
| | sulfones | |
| | Acrylate and ortho-nitrobenzyl | Blue light |
| | Adamantan and beta-cyclodextrin | Ultrasound |
| PVP | N,N-methylene bis acrylamide | Two-photon laser |
| | (NBS) | |
| Polymethyl | Ethylene glycol dimethacrylate and | Two-photon laser |
| methacrylate | alkaline hydrolysis | |
| Polyvinyl alcohol | Acylates, acrylamides | Two-photon laser |
| | Adamantan and beta-cyclodextrans | Ultrasound |
| | Thiol and 3,5-dichloro-1,2,4,5 | UV and Visible Light |
| | tetrazine | |
| Poly(acrylic) acid | Azo groups and beta-cyclodextrans | Red light |
| Polyacrylamide | NBS | Two-photon laser and ultrasound |
| | Azo groups, N,N- | UV light |
| | methylenebisacrylamide | |

A particular handheld instrument could be used or developed to produce ultrasound waves or UV light, or a two-photon laser with the intent of dissolving the microparticles by applying it to the surface of the skin. Various existing light wands are currently available.

### Shelf Life of Microbeads

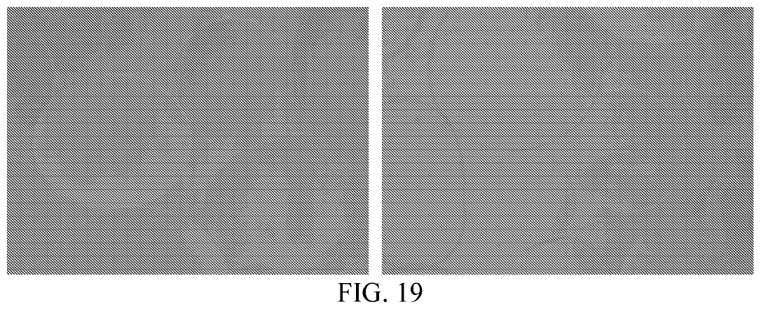

Informally, we have maintained PEG-based hydrogel microbeads at room temperature in PBS for over 4 years with no visible deterioration of the physical properties. FIG. 19 provides two example images of thiolated hyaluronic acid stored at room temperature in water for more than four years and then imaged. More formally, we have cryopreserved microspheres and determined their structural integrity following thawing.

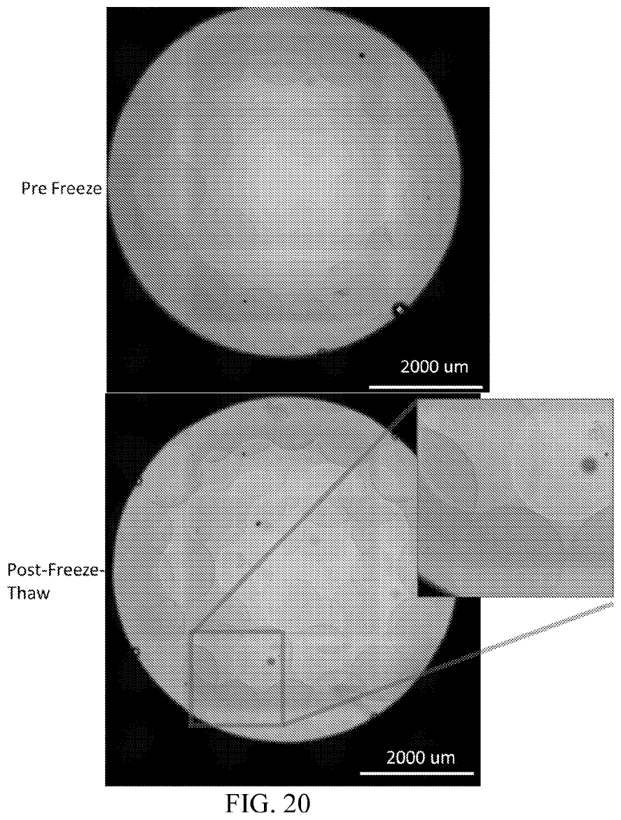

The final crosslinked product can be frozen without negatively affecting the chemistry of the physical properties of the microparticles using a slow-freeze protocol and found that upon thawing by placing at 37 C for approximately 2 minutes, the microparticles maintain their structure and surface topography. FIG. 20 illustrates pictures of the microparticles before cryopreservation at −80° C. and after cryopreservation and thawing with no change in size, shape or microscopic structure.

### Sterilization of Microparticles

Manufacturing of the microparticle using the CSS method is completely closed and sterile. However, manufacturing by other means such as emulsion does not lend itself easily to sterile conditions. In such cases, the final product may require sterilization. Table 7 provides the results of sterilization experiments in which microparticles in their final format were autoclaved.

TABLE 7
Physical Properties of Microparticles after Sterilization

| | HA | | PVA | |
|---|---|---|---|---|
| | Pre-Sterilization | Post-Sterilization | Pre-Sterilization | Post-Sterilization |
| Diameter (μm) | 836.9 ± 4.7 | 837.4 ± 4.4 | 818.0 ± 5.1 | 805.6 ± 5.2 |
| Polymer Mass | 25.7 ± 0.1 | 28.5 ± 0.1 | 21.0 ± 0.1 | 22.1 ± 0.2 |
| Fraction (Q) | | | | |

1. A method for aesthetic or cosmetic soft tissue filling in a human subject, said method comprising placing into a site of implantation in a subject in need thereof, a suspension comprising a plurality of porous viscoelastic solid hydrogel microparticles dispersed in a carrier vehicle, wherein each microparticle is a self-sustaining body having an outer surface and a defined/discrete 3-dimensional shape and geometry, wherein said microparticles are substantially free of any cells, tissue, or therapeutic compounds.

2. The method of claim 1, wherein said suspension is placed into the superficial dermal layers, mid-dermal layers, sub-dermal/subcutaneous layers, facia, muscle, or periosteal layers of said subject.

3. (canceled)

4. The method of claim 1, wherein said suspension is placed into said site of implantation via injection.

5. The method of claim 4, wherein said suspension is injected through an injection device having an opening smaller than a particle size of the microparticles wherein said microparticles fracture upon said injection.

6. The method of claim 1, wherein said hydrogel microparticles are characterized as self-sustaining bodies having a 3-dimensional matrix of covalently or ionically crosslinked polymer compounds.

7. The method of claim 6, wherein said polymer compounds are selected from the group consisting of hyaluronic acid, polyethylene glycol, hyaluronan, fibrin, chitosan, collagen, heparin, polylactic acid (PLA), poly(L-lactic acid) (PLLA), polylactic-co-glycolic acid (PLGA), polycaprolactone (PCL), polyvinyl alcohol (PVA), polyvinylpyrrolidone (PVP), methacrylic acid (MAA), 2-hydroxyethyl methacrylate (HEMA), polyacrylamide (PAM) extracellular matrix, and functionalized varieties of the same.

8. The method of claim 1, wherein said defined/discrete 3-dimensional shape and geometry is a rounded shape and geometry having a sphericity Ψ of greater than 0.25.

9. The method of claim 1, wherein said hydrogel microparticles have a particle size of greater than 30 μm and less than 2 mm.

10. The method of claim 1, wherein said hydrogel microparticles: induce a favorable physiological response, increase amount or quality of tissue volume, or provide a resilient response to produce a natural appearance in the subject at the site of implantation.

11.-12. (canceled)

13. The method of claim 1, wherein the hydrogel microparticles have a first particle size in said suspension, wherein said microparticles expand after introducing into the site of implantation, wherein said microparticles have a second particle size in said site of implantation that is greater than said first particle size.

14. The method of claim 1, said suspension further comprising a solute in suspension with said microparticles, wherein said solute causes said microparticles to contract in said suspension, such that the microparticles have a first particle size in said suspension, wherein said microparticles have a second particle size in said site of implantation that is greater than said first particle size due to decreasing concentration of said solute after said placement in said site of implantation.

15. The method of claim 1, said plurality of porous viscoelastic solid hydrogel microparticles comprise at least two different populations of microparticles, wherein a first population of microparticles comprises a plurality of microparticles having a first characteristic, and a second population of microparticles comprises a plurality of microparticles having a second characteristic, wherein said first characteristic is different from said second characteristic.

16. The method of claim 1, wherein said delivery vehicle is a buffered saline solution, viscous solution, putty, paste, or gel, optionally containing an anesthetic, antibiotic, or analgesic.

17.-21. (canceled)

22. The method of claim 1, further comprising dissolving said microparticles in the site of implantation by introducing a chemical or enzyme into the site of implantation.

23. The method of claim 1, further comprising dissolving said injected microparticles in the site of implantation by introducing a physical stimulation such as sound waves or UV light into the site of implantation.

24. A kit for cosmetic or aesthetic soft tissue fillers, comprising a plurality of porous elastic solid hydrogel microparticles wherein each microparticle is a self-sustaining body having an outer surface and a defined/discrete 3-dimensional shape and geometry, and substantially free of any cells, tissue, or therapeutic compounds, and instructions for administering the same.

25. The kit of claim 24, wherein said plurality of porous elastic solid hydrogel microparticles are lyophilized, said kit further comprising a carrier vehicle in a separate container for reconstituting said hydrogel microparticles or instructions for reconstituting said hydrogel microparticles prior to administration.

26. The kit of claim 25, further comprising a syringe and instructions for loading said syringe with said hydrogel microparticles after reconstitution.

27. The kit of claim 24, wherein said plurality of porous viscoelastic solid-like hydrogel microparticles are packaged in suspension, wherein said suspension is pre-loaded into a syringe for administration and instructions for the same.

28. The kit of claim 24, wherein said plurality of porous viscoelastic solid-like hydrogel microparticles are packaged in suspension wherein said kit comprises an empty syringe and said hydrogel microparticles in a separate container configured to permit withdrawal of the suspension from the container to load said syringe prior to said administration, and instructions for the same.

29. The kit of claim 24, further comprising a separate container comprising a dissolution chemical or enzyme for removal of the implanted viscoelastic solid-like hydrogel microparticles on demand.

30. The kit of claim 24, further comprising a handheld device to dissolve the implanted viscoelastic solid-like hydrogel microparticles on demand via application of light, sound, or ultrasonic energy.

31. The kit of claim 28, wherein said plurality of porous viscoelastic solid-like hydrogel microparticles are packaged in a storage suspension comprising a solute, wherein said solute causes said microparticles to contract in said suspension, such that the microparticles have a first particle size in said storage suspension that is smaller than the particle size of said microparticles after administration to a subject.

32.-33. (canceled)
